(19) United States
(12) Patent Application Publication
(10) Pub. No.: US 2015/0284587 A1
(43) Pub. Date: Oct. 8, 2015

(54) COMPOSITION OF AND METHOD FOR FORMING REDUCED VISCOSITY POLYMERIC COATINGS

(71) Applicant: BACTERIN INTERNATIONAL, INC., Belgrade, MT (US)

(72) Inventors: Mark Schallenberger, Bozeman, MT (US); Ben Luchsinger, Bozeman, MT (US); Todd Meyer, Bozeman, MT (US)

(21) Appl. No.: 14/379,199

(22) PCT Filed: Feb. 19, 2013
(86) PCT No.: PCT/US13/26710

§ 371 Date: Aug. 15, 2014

Related U.S. Application Data
(60) Provisional application No. 61/600,453, filed on Feb. 17, 2012.

(51) Int. Cl. C09D 167/04; A61B 17/68; A61M 25/00; A61F 2/02; A61M 27/00; A61N 1/362; A61F 2/82; C08K 5/29; A61M 16/04

(52) U.S. Cl. CPC C09D 167/04; C08K 5/29; A61B 17/68; A61M 25/0009; A61M 16/04; A61M 27/00; A61N 1/362; A61F 2/82; A61F 2/02; A61B 2017/00889

(57) ABSTRACT

The present invention is directed to polymeric coating formulations in which addition of an antimicrobial to a solution, containing solvent and polymer, significantly alters polymer entanglements. Benefits include alternative routes to, and customization of the polymer's degradation pathways. The dramatic entanglement changes also provide manufacturing advantages such as ease of stirring, pumping, and spraying. Furthermore, the coatings may have faster drying times, less shrinkage, more complete fill, and a more even coat for a given thickness. Methodology for production of coating formulations of the desired properties and their use to provide antimicrobial coatings is also described.

### CROSS REFERENCE TO RELATED APPLICATIONS

This application claims priority under 35 U.S.C. §119(e) to U.S. Provisional Patent Application Ser. no. 61/600,453 on Feb. 17, 2012, which is incorporated herein in its entirety by reference.

### FIELD OF THE INVENTION

This invention relates to an antimicrobial polymeric coating constructed using a polymer solution where the polymer entanglements are significantly decreased by the presence of antimicrobial/viscosity reducing agents.

### SUMMARY OF THE INVENTION

For patients with an implanted medical device, implant-associated infections remain a significant risk. In order to minimize the risk of bacterial and fungal-related illness a variety of antimicrobial/bioactive agents have been employed at home and in the clinical setting. Although selected agents have proven abilities to limit disease and inhibit microbial growth, there remains a need for improved infection control. An object of this invention is the use of antimicrobial coatings for a variety of substrate surfaces (metals, plastics, glasses, elastomers, and others) with a particular emphasis on the coating of medical devices.

While several methods of coating medical devices with bioactive agents exist, it remains particularly advantageous to obtain coatings with improved properties of uniformity and consistency. It is therefore commercially desirable to provide a coating formulation with adjustable physical properties to allow modification of the resultant antimicrobial film.

The present invention is directed to coating compositions used to generate antimicrobial-coated medical devices. A preferred embodiment of the present invention comprises polymeric coating formulations where the polymer entanglements are significantly decreased by the presence of viscosity reducing agents. A further embodiment includes a change in the crystalline structure within the polymer matrix. Potential advantages to be gained from these embodiments include alternative routes to, and customization of, the polymer's degradation pathways.

Decreases in polymer entanglements and solution viscosity may yield a number of manufacturing advantages and embodiments. Polymer solutions containing viscosity reducers may produce coatings with the same thickness as corresponding polymer-only solutions, but with much lower viscosity. These coating formulations may provide a more even coating for a given film thickness. In addition, less viscous polymer solutions may exhibit faster drying times, less shrinkage, more complete fill, and fewer air pockets in complex molds. Furthermore, due to the decrease in solution viscosity the polymer solutions may be more effectively stirred, mechanically pumped, and sprayed.

### BRIEF DESCRIPTION OF THE DRAWINGS

FIG. 1 provides a detailed explanation of lactide-glycolide polymer nomenclature.

FIG. 2 presents a graphical representation of the film thickness for coating formulations of PLGA with and without CHX present.

### DETAILED DESCRIPTION OF THE INVENTION

One aspect of the present invention describes an antimicrobial coating composed of a biodegradable polymeric carrier and an antimicrobial agent, which reduces the viscosity of the coating formulation. This formulation may be used to coat surfaces of medical devices to inhibit microbial growth and/or colonization. An embodiment of the present invention comprises antimicrobial polymer coatings, films, and molds, where the polymer entanglements are significantly decreased by the presence of bioactive agents. Polymer entanglement may be significantly decreased by the antimicrobial agent within the coating. A further embodiment includes antimicrobial polymer coatings, films, and molds having a change in the crystalline structure within the polymer matrix. Advantages from these embodiments include alternative routes to, and customization of, the polymer's degradation pathways.

Not wishing to be bound by theory, it is thought that the crystalline nature of the film may increase by allowing less tangled polymer chains to line up, or decrease by increasing the amount of disorder in the coating after the changes have had less entanglement.

“Coating,” as used herein, broadly includes any film, layer, or mold that covers or partially covers a surface.

“Viscosity,” as used herein, refers to the property of a fluid to resist the force tending to cause the fluid to flow. In the context of polymeric solutions, increased polymeric entanglement leads to increased viscosity.

Within a polymeric matrix, polymer chains entangle with one another. “Entanglement,” as used herein, refers to the degree that polymer chains interact with each other within a solution, gel, or solid. In relation to the current invention, the higher the degree of polymer chain entanglement within a solution, the more viscous the solution.

One aspect of the present invention describes a method of preparing a polymeric coating on a medical device. In general, as polymers are added to solvents the polymers raise the viscosity of the resulting coating formulation proportional to the polymers concentration in the solvent. In certain circumstances, this increased solution viscosity may be detrimental to the manufacturing process. Adding an antimicrobial agent, which acts as a viscosity reducing agent(s), even in small amounts, to a solution comprising a polymer reduces the coating formulation viscosity thereby reducing or eliminating viscosity-related complications. Due to the decrease in solution viscosity, coating formulations containing an antimicrobial agent that acts as a viscosity reducer are more effectively stirred, mechanically pumped, and sprayed. Additionally, less viscous coating formulations provide a more even dip coating than equivalent, yet more viscous, coating formulations. Alternatively, coating formulations containing an antimicrobial agent that acts as a viscosity reducer provide greater film thickness than equivalent coating formulations without antimicrobial agents of the same viscosity because for a solution of a given viscosity, it is possible to have a higher concentration of polymer in the solution, thereby obtaining a thicker coating.

Polymers or copolymers which may be used with different aspects and embodiments of this invention include any polymer type but in the provided embodiments specifically relate to biodegradable, biocompatible polymers. Polymers may include, but are not limited to, polycaprolactones, polyethylene glycols, polyhydroxyalkanoates, polyesteramides, polylactides, polyglycolides, poly(lactide-co-glycolide)s, polyorthoesters, polyoxazolines, polyurethanes, or copolymers. Polymers may be used by themselves or in combination with other polymers.

In embodiments of the present invention, the polymer may be poly(lactide-co-glycolide), hereafter referred to as PLGA. PLGA may contain different concentrations of lactide and glycolide. In some embodiments, the PLGA comprises between about 10% to about 90% of lactide and about 90% to about 10% of glycolide. PLGAs comprising poly(D,L-lactide-co-glycolide) with about 75% of D,L-lactide and about 25% of glycolide.

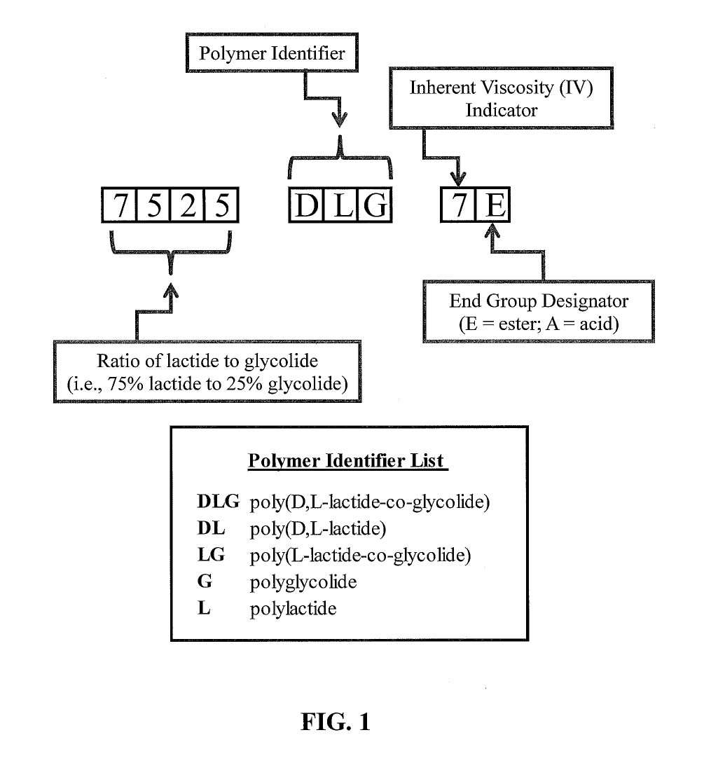

Another copolymer of the invention is PLGA composed of poly(L-lactide-co-glycolide). The average molecular weight of the PLGA and the ratio of lactide to glycolide may be varied to tailor the mechanical, physiochemical, and biodegradable properties of the polymer to the desired ranges. The nomenclature system for lactide-glycolide copolymers is detailed in FIG. 1. By way of example, FIG. 1 illustrates a polymer material comprising lactide and glycolide. The first numbers, 7525, represent the amount of lactide to glycolide in the polymer—about 75% lactide to about 25% glycolide. The polymer identifier identifies the polymeric material—DLG is poly(D,L-lactide-co-glycolide); D,L is poly(D,L-lactide); LG is poly(L-lactide-co-glycolide); G is polyglycolides and L is polylactide. The Inherent Viscosity (IV) indicator is proportional to the molecular weight of the polymer. The IV values are derived from viscosity measurements of a solution of the polymer at about 0.5% w/v in CHCl₃ at about 30 ° C. For a polymer of the name 7525 DLG 7E, the second seven indicates an IV range of about 0.6 dL/g to about 0.8 dL/g. A larger inherent viscosity indicates a higher molecular weight polymer. In some embodiments, the PLGA may have an IV range of between about 0.10 dL/g to about 1.0 dL/g. In some embodiments, the PLGA may have an IV range of between about 0.4 dL/g to about 0.8 dL/g.

In the various compositions of the present invention, the polymer material may comprise between about 1% by weight to about 30% by weight, about 5% by weight to about 25% by weight, or about 10% by weight to about 15% by weight to volume of the carrier solvent.

Various aspects and embodiments of the present invention include a viscosity reducing agent. The viscosity reducing agent may be an antimicrobial agent. In some embodiments, the antimicrobial agents may be, but are not limited to, biguanides such as chlorhexidine, 1,1′-hexamethylene-bis(5-[2-ethylhexyl]biguanide, polyaminopropyl biguanide, polyhexamethylene biguanide, salts thereof, and combinations thereof. Biguanide salts usable for the present invention may include inorganic or organic counterions such as acetate, bromide, carbonate, chloride, citrate, fumarate, mesylate, phosphate, propionate, succinate, sulfate, sulfonate, and tartrate, In the present invention, chlorhexidine may be used as its free base, hereafter referred to as CHX.

In various embodiments, the viscosity of compositions with the viscosity reducing agent may have a significantly reduced viscosity compared to solutions without the viscosity reducing agent. In some embodiments, the viscosity may be reduced by at least about 10%, at least about 20%, at least about 30%, at least about 40%, at least about 50%, at least about 60%, at least about 70%, at least about 80% and at least about 90% when the viscosity is measured in any conventional manner.

In the various compositions of the present invention, the antimicrobial agent may comprise between about 0.01% by weight to about 50% by weight, about 2% by weight to about 25% by weight, or about 5% by weight to about 15% by weight to weight of the polymer. In some embodiments, the antimicrobial agent may be about 25% by weight to weight of the polymer.

In the various embodiments of the present invention, the polymeric carrier and an antimicrobial agent that acts as a viscosity reducer may be combined and mixed with a carrier solvent. Suitable carrier solvents may be any solvent, combination of carrier solvents or a mixture containing the carrier solvent in which the polymeric material and the antimicrobial agent(s) are at least partially soluble, and in some embodiments, are fully soluble. Carrier solvents may include, but are not limited to, acetone, acetonitrile, chloroform, diethyl ether, dimethylacetamide, dimethylformamide, dimethylsulfoxide, ethanol, ethyl acetate, hexafluoroisopropanol, hexane, methanol, methylene chloride, tetrahydrofuran, toluene, water and combinations thereof.

In some embodiments, the coating is between about 0.1 microns to about 500 microns thick. In other embodiments, the coating may be about 1 micron to about 15 microns. In a further embodiment, the coating may be about 2 microns to about 5 microns.

In various aspects and embodiments of the present invention, bioactive agents may be included in the coating formulation and resulting coating. Bioactive agents including, but not limited to, antibiotics, antimicrobials, biocompatible minerals, cells, growth factors, hormones and combinations thereof. Additional antimicrobials of interest for the coating formulations include, but are not limited to, silver nanoparticles, silver nitrate, silver oxide, silver salts, silver sulfadiazine, silver zeolites, triclosan or combinations thereof. A variety of antibiotics may be additionally incorporated into the coating including, but not limited to, antifolates, aminoglycosides, carbapenems, cephalosporins, fluoroquinolines, glycopeptides, macrolides, monobactams, oxazolidones, penicillins, rifamins, sulfonamides tetracyclines or combinations thereof. Antibiotics of particular interest for inclusion within the films include, but are not limited to, clindamycin, gentamicin, minocycline, rifampin, tobramycin, and vancomycin or combinations thereof. Suitable biocompatible minerals for coating incorporation include, but are not limited to, bioglasses, hydroxyapatites, phosphates, sulfates or combinations thereof. The foregoing bioactive agents may be added to the coating formulation individually or as a mixture of multiple bioactive components.

An aspect of the present invention is a coating formulation. The coating formulations of the present invention include the polymeric material, an antimicrobial agent that acts as a viscosity reducer, and a carrier solvent. Polymer entanglement may be significantly decreased by the antimicrobial agent within the coating formulation. Additionally, the crystalline polymer structure and/or the amorphous polymer structure within the polymer material may be altered. The coating formulation may further comprise at least one bioactive agent. The antimicrobial agent of the present invention may be a biguanide.

Another aspect of the present invention comprises a method for making the coating with reduced viscosity. A coating formulation is generated by adding the polymer and an antimicrobial agent that acts as a viscosity reducer into a carrier solvent or carrier solvent mixture. The resultant mixture is agitated at between about 0° C. to about 75° C., in some embodiments about 40° C. until at least a portion, if not all, of the solids present in the mixture are dissolved. The coating formulation is cooled to between about 0° C. to about 50° C., in some embodiments about 22° C.

In some embodiments, the resultant mixture may be agitated by any suitable method. The agitation step may be performed by mechanical stirring, magnetic stirring, ultrasonication, shaking, homogenizing, vortexting, or combinations thereof.

Some embodiments further include a method to coat an article. An article may be coated in the coating formulation, and after removal of the article from the coating formulation, the carrier solvent is evaporated from the article at temperatures between about 0° C. to about 50° C., in some embodiments under ambient conditions, for a sufficient period to substantially evaporate the carrier solvent. In some embodiments, the carrier solvent may be evaporated for between about 1 second to about 96 hours, in some embodiments between about 24 hours to about 48 hours, depending upon the evaporation conditions. The pressure during evaporation may be ambient or reduced pressure.

Articles to be coated may include metal articles. Metal articles may be pre-treated by various standard methods (e.g., acid etching, sonication, and passivation). Other suitable materials for the articles include, but are not limited to, plastics, elastomers, glasses, tissues, and combinations thereof.

Articles may be coated by submersion into the coating formulation followed by withdrawal from the coating formulation at a controlled rate. In some embodiments, the controlled rate is between about 0.1 cm/sec to about 10 cm/sec. In some embodiments, the controlled rate is about 1.0 cm/sec. Alternatively, the coating formulation may be applied using any suitable method including, but not limited to, dipping, submersion, spraying, painting, and combinations thereof. In some embodiments, the applying step may be a more rapid application, such as a dip, wherein the rapid dip coating rates may be achieved for a coating thickness. In some embodiments, the coating may be applied evenly on the medical device. In some embodiments, the coating may dry faster than a coating without the antimicrobial agent. In still other embodiments, the shrinkage of the coating formulation may be reduced compared to coating formulations without the antimicrobial agent. In still other embodiments, air pockets may be reduced in the coating compared to coating formulations without the antimicrobial agent.

Articles may be a medical device selected from the group consisting of orthopedic implants, catheters, endotracheal tubes, wound drains, pacemakers, portacaths, stents, any other medical device manufactured from metal, glasses, tissue, elastomers, plastics, and combinations thereof. Specific examples of medical devices include an implantable medical device, an orthopedic device, an implantable orthopedic device, an orthopedic screw, a K-wire, an implantable tissue, and a bone substitute, and combinations thereof.

An aspect of the invention is a method for coating a medical device comprising casting the coating formulation having a low viscosity on the medical device, then evaporating the solvent of the coating formulation to form a coating on the device. The method for coating the medical device may include adding bioactive agents.

Another aspect of the invention is a coating, wherein the coating comprises a polymeric material and an antimicrobial agent, wherein the antimicrobial agent lowers the viscosity of a coating formulation. The coating may further include at least one bioactive agent.

Another aspect of the present invention comprises a coated medical device. The coated medical device comprises a medical device and a coating on the medical device, wherein the coating comprises a biodegradable polymeric material and an antimicrobial agent, wherein the antimicrobial agent has lower the viscosity of a coating formulation. The coating may further include at least one bioactive agent.

### EXAMPLE 1

### PLGA Films

The following method was used to produce a coating solely composed of PLGA. Acetonitrile (CH₃CN) (14.0 mL) was added to PLGA (7525 DLG 7E, 700 mg, 5% w/v) with stirring. The resultant mixture was stirred at about 40° C. until the PGLA substantially dissolved in the solvent. The solution was then allowed to cool to about 22° C. Articles were then coated by submersion into the coating solution followed by withdrawal from the solution at a controlled rate. After removal from the coating solution the casting solvent was allowed to evaporate from the articles under ambient conditions for between about 24 hours to about 48 hours.

### EXAMPLE 2

### PLGA and CHX Films

The following method was used to provide a coating composed of PLGA and CHX. PLGA was the polymer material (7525 DLG 7E, 2.1 g, 15% w/v) and the free base form of CHX (0.210 g, 10% w/w) were added to a stirring solution of CH₃CN (14.0 mL). The resultant mixture was stirred at about 40° C. until all solids dissolved. The solution was then allowed to cool to about 22° C. Articles were then coated by submersion into the coating solution followed by withdrawal from the solution at a controlled rate. After removal from the coating solution the casting solvent was allowed to evaporate from the articles under ambient conditions for about 24 hours to about 48 hours.

### Effects of CHX on Coating Thickness

To obtain a comparison of the effects of CHX on coating thickness, a series of coating solutions were prepared. The thicknesses of the coatings were determined by inclusion of crystal violet (a dye) within the coatings at a concentration of 0.5% weight to weight of the PLGA. The quantity of dye present within the cast films was determined by measurement of the UV absorbance of dissolved coatings. The thickness of the coating was calculated based on the ratio of polymer to the dye, surface area of the coating, and polymer density.

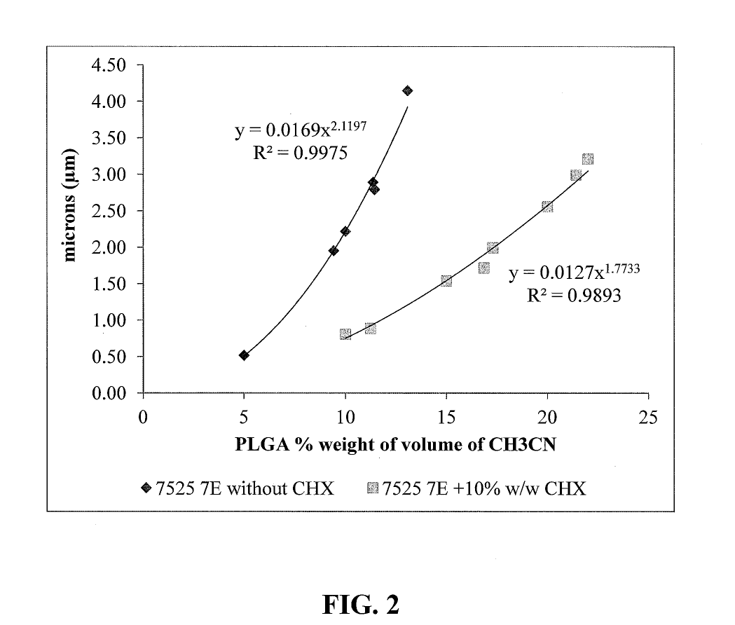

Several formulations were prepared incorporating only PLGA (7525 DLG 7E) and crystal violet. Coatings were prepared as described in Example 1 using varying amounts of PLGA and the addition of crystal violet. The coating compositions are detailed in Table 1 alongside the calculated coating thicknesses. For comparison, an additional series of formulations were prepared with the addition of chlorhexidine at about 10% weight to weight of the PLGA. These formulations were prepared as described in Example 2 using varying amounts of PLGA and CHX and the addition of crystal violet. The coating compositions are detailed in Table 2 alongside the calculated coating thicknesses. The results of this survey are presented in FIG. 2. The addition of CHX to the polymer matrix provides notably thinner films. These results are an expected consequence of the reduced viscosity of the coating solutions.

TABLE 1
Coating Thickness in the Absence of Chlorhexidine

| | Quantity of | | Quantity of | Calculated |
|---|---|---|---|---|
| | PLGA | Polymer | Crystal Violet | Coating |
| | (7525 DLG 7E) | % | (0.5% w/w of | Thickness |
| Coating | (mg) | w/v | PLGA) (mg) | (μm) |
| 1 | 250 | 5.00 | 1.25 | 0.52 |
| 2 | 471 | 9.41 | 2.35 | 1.95 |
| 3 | 500 | 10.00 | 2.50 | 2.22 |
| 4 | 568 | 11.35 | 2.84 | 2.89 |
| 5 | 572 | 11.43 | 2.86 | 2.79 |
| 6 | 654 | 13.07 | 3.27 | 4.15 |

TABLE 2
Coating Thickness in the Presence of Chlorhexidine

| | Quantity | | | | |
|---|---|---|---|---|---|
| | of PLGA | | Quantity of | Quantity of | Calculated |
| | (7525 | | Crystal Violet | Chlorhexidine | Coating |
| Coat- | DLG 7E) | Polymer | (0.5% w/w of | (10% w/w of | Thickness |
| ing | (mg) | % w/v | PLGA) (mg) | PLGA) (mg) | (μm) |
| 1 | 500 | 10.00 | 2.50 | 50 | 0.83 |
| 2 | 560 | 11.20 | 2.80 | 56 | 0.89 |
| 3 | 750 | 15.00 | 3.75 | 75 | 1.54 |
| 4 | 845 | 16.90 | 4.23 | 85 | 1.72 |
| 5 | 865 | 17.30 | 4.33 | 87 | 2.00 |
| 6 | 1000 | 20.00 | 5.00 | 100 | 2.56 |
| 7 | 1070 | 21.40 | 5.35 | 107 | 2.99 |
| 8 | 1100 | 22.00 | 5.50 | 110 | 3.21 |

### Comparison of Formulation Viscosity and Thickness

The addition of CHX to a solution of PLGA in solvent causes a significant change in solution viscosity. All viscosity measurements were obtained by use of a Cannon-Finske Opaque Viscometer at 22° C. For a coating solution prepared by the method of Example 1 the kinematic viscosity is 2.78 cSt (Table 3, Formulation A). To obtain a solution of the same kinematic viscosity in the presence of CHX requires a substantial increase in the quantity of PLGA. A coating solution prepared by the method of Example 2 provides a solution with a kinematic viscosity at 2.95 cSt (Table 3, Formulation B). Thus in this comparison, approximately three times the amount of PLGA is needed to obtain matching solution viscosities. Although the formulations have similar viscosities the coating thickness is directly proportional to the concentration of PLGA within the dipping solution. As a result, formulation B with three times the amount of PLGA as formulation A also has a coating thickness of three times the thickness of formulation A. As described in the proceeding section, coating thicknesses were determined by the addition of crystal violet to coating solutions followed by film casting and subsequent UV absorbance measurements.

TABLE 3
Formulation Comparison

| | Amt. | | | | Calculated |
|---|---|---|---|---|---|
| | 7525 DLG | | Amt. | Kinematic | Coating |
| For- | 7E PLGA | Amt. CHX | CH₃CN | Viscosity | Thickness |
| mulation | (g) | (g) | (mL) | (cSt) | (μm) |
| A | 0.700 | 0 | 14.0 | 2.78 | 0.51 |
| B | 2.100 | 0.210 | 14.0 | 2.94 | 1.57 |

### EXAMPLE 4

Example 4 illustrates the effect of chlorhexidine as a viscosity reducing agent. Two different solutions of PLGA 7525 DLG 7E in acetonitrile were prepared as shown in Table 4. The amount of chlorhexidine, which was used as a viscosity reducing agent, was the only variable between the two samples. The resulting kinematic viscosity was much lower for the solution containing the chlorhexidine (Solution 2) compared to the solution that did not contain any chlorhexidine.

TABLE 4

| | Amt. | | Amt. | Kinematic |
|---|---|---|---|---|
| | PLGA (g) | Amt. CHX (g) | CH₃CN | Viscosity |
| Formulation | (15% w/v) | (0 or 10% w/w) | (mL) | (cSt) |
| Solution 1 | 2.1 | 0 | 14 | 26.6 |
| Solution 2 | 2.1 | 0.21 | 14 | 2.94 |

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1-25. (canceled)

26. A method for coating a medical device comprising:
i. casting upon the medical device a coating formulation having low viscosity, comprising:
a. a polymeric material;
b. an antimicrobial agent, wherein the antimicrobial agent reduces the viscosity of the coating formulation; and
c. a carrier solvent;
ii. applying the coating formulation to the medical device; and
iii. evaporating the solvent from the coating formulation to form a coating on the medical device.

27. The method of claim 26, wherein the step of applying the coating formulation is selected from the group consisting of dipping, submersion, spraying, painting, and combinations thereof.

28-30. (canceled)

31. The method of claim 26, wherein the medical device is selected from the group consisting of orthopedic implants, catheters, endotracheal tubes, wound drains, pacemakers, portacaths, stents, elastomeric, and polymeric implants.

32-41. (canceled)

42. The method of claim 26, wherein the polymeric material of the coating formulation is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

43. The method of claim 26, wherein the solvent of the coating formulation is selected from the group consisting of acetone, acetonitrile, chloroform, diethyl ether, dimethylacetamide, dimethylformamide, dimethylsulfoxide, ethanol, ethyl acetate, hexafluoroisopropanol, hexane, methanol, methylene chloride, tetrahydrofuran, toluene, water, and combinations thereof.

44. (canceled)

45. The method of claim 26, wherein the antimicrobial agent of the coating formulation is selected from the group consisting of biguanides, antifolates, aminoglycosides, carbapenems, cephalosporins, fluoroquinolines, glycopeptides, macrolides, monobactams, oxazolidones, penicillins, rifamins, sulfonamides, tetracyclines, clindamycin, gentamicin, minocycline, rifampin, tobramycin, vancomycin, silver nanoparticles, silver nitrate, silver oxide, silver salts, silver sulfadiazine, silver zeolites, triclosan, and combinations thereof.

46. The method of claim 26, wherein the coating is applied evenly on the medical device.

47. The method of claim 26, wherein the coating dries faster than a coating formulation without the antimicrobial agent.

48. The method of claim 26, wherein a shrinkage of the coating is reduced compared to coating formulations without the antimicrobial agent.

49. The method of claim 26, wherein air pockets are reduced in the coating compared to coating formulations without the antimicrobial agent.

50-57. (canceled)

58. A coating, comprising:
a. a biodegradable polymeric material; and
b. an antimicrobial agent, wherein the antimicrobial agent lowers the viscosity of a coating formulation.

59. The coating of claim 58, wherein the polymeric material is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

60. The coating of claim 58, wherein the polymeric material is poly(lactide-co-glycolide).

61-65. (canceled)

66. The coating of claim 58, wherein an amount of the polymeric material in the coating is between about 90% and about 50% by weight to volume of the coating.

67-68. (canceled)

69. The coating of claim 58, wherein the antimicrobial agent is chlorhexidine.

70-75. (canceled)

76. The coating of claim 58, wherein the antimicrobial agent is present in the coating in amounts between about 0.01% to about 50% by weight of the polymeric material.

77-79. (canceled)

80. A coated medical device, comprising:
a. a medical device; and
b. a coating on the medical device, wherein the coating comprises:
i. a biodegradable polymeric material; and
ii. an antimicrobial agent, wherein the antimicrobial agent lowers the viscosity of a coating formulation.

81. The medical device of claim 80, wherein the polymeric material of the coating is selected from the group consisting of polycaprolactones, polyethylene glycols, polyhydroxyalkanoates, polyesteramides, polylactides, polyglycolides, poly(lactide-co-glycolide)s, polyorthoesters, polyoxazolines, polyurethanes and combinations thereof.

82. The medical device of claim 80, wherein the polymeric material of the coating is poly(lactide-co-glycolide).

83-84. (canceled)

85. The medical device of claim 80, wherein the antimicrobial agent is chlorhexidine.

86. (canceled)
